(19) United States
(12) Patent Application Publication
(10) Pub. No.: US 2022/0306749 A1
(43) Pub. Date: Sep. 29, 2022

(54) Use of an Anti-P-selectin Antibody

(71) Applicant: NOVARTIS AG, Basel (CH)

(72) Inventors: Dram LAINE, East Hanover, NJ (US); Savita NANDAL, Livingston, NJ (US); Jincy PAULOSE, Stony Point, NY (US); Das PURKAYASTHA, East Hanover, NJ (US); Cuong TRUONG, Pomona, NY (US)

(21) Appl. No.: 17/616,900

(22) PCT Filed: Jun. 5, 2020
(86) PCT No.: PCT/US2020/036221

§ 371 Date: Dec. 6, 2021

Related U.S. Application Data
(60) Provisional application No. 62/858,526, filed on Jun. 7, 2019.

(51) Int. Cl. C07K 16/28; A61P 15/00

(52) U.S. Cl. CPC C07K 16/2854; A61P 15/00; A61K 2039/505

(57) ABSTRACT

The invention relates to a method of treating priapism in a patient in need of such treatment, comprising administering a pharmaceutically effective amount of an anti-P-selectin antibody or a binding fragment thereof to said patient, especially wherein the patient is suffering from Sickle Cell Disease (SCD), and related invention embodiments (uses, methods, pharmaceutical preparations and use in the preparation of pharmaceutical preparations).

### FIELD OF INVENTION

The invention relates to the treatment of priapism, especially in patients with Sickle cell Disease (SCD), with a drug, especially an antibody, capable of binding to P-selectin. Methods of treatment, uses, pharmaceutical preparations, their manufacture and the drug for use in treatment of priapism, respectively, are examples of specific embodiments of the invention.

### BACKGROUND

Priapism is an involuntary, painful and persistent penile erection that lasts longer than normal and that is found without relationship to sexual activity.

Three types of priapism have been differentiated: (1) ischemic, (2) recurrent ischemic (“stuttering priapism”) and (3) non-ischemic priapism. The vast majority of cases are ischemic.

This invention is mainly related to ischemic and recurrent ischemic priapism.

Profoundly long priapism may lead to irreversible damage to the penis of male patients. Therefore, various treatment methods have been suggested which partially are also very cumbersome for the affected patients.

In acute phase priapism, it is recommended that treatment is started with aspiration of blood and/or injection of an alpha-adrenergic agent.

For long term treatment or in the case the acute phase treatment fails, shunt procedures allowing for increased blood flow are a commonly used procedure. In the case of recurrent priapism, androgen ablation therapy was regarded as effective, or, in the case of men with sickle cell disease (SCD), treatment with 5-phosphodiesterase inhibitors. For a review see Matthew Hudnall et al., Trans. Androl. Urol. 2017, 6(2), 199-206.

In this review and the literature cited therein, among the mechanisms forming a basis for priapism, PDE5 regulation and subsequent aberrant NO signaling has been described to be an important cause of stuttering priapism.

Roghmann et al. also refers to other signaling pathways involving adenosine, RhoA/rock and opiorphin that may also be involved in the pathogenesis of priapism.

Preventive treatments to reduce the number of vaso-occulusion crisis (VOCs) are limited. Hydroxyurea/hydroxycarbamide (HU/HC) is approved to reduce the frequency of painful crises and the need for transfusions in SCD patients aged 2 years and older with a history of recurrent, moderate-to-severe painful crises.

Another therapy, L-glutamine (Endari™), is approved in the United States to reduce the acute complications of SCD in adult and pediatric patients 5 years and older (Niihara et al 2018).

Especially, neither transfusion nor HU/HC or L-glutamine are working against priapism in SCD patients.

Also, phenylephrine injection at home often does not work.

In the case of SCD the vast majority of cases are ischemic, in which increased pressure, compromises the vascular circulation (i.e. a type of compartment syndrome). Its ischemic form, presenting as a single, major event, or recurrence, is associated with penile pain, erectile tissue destruction and loss, and permanent erectile inability. The sickled erythrocytes predispose to venous stasis, which therefore perpetuates the priapism. Cavernosography shows that this stasis results in obstruction of the deep dorsal penile vein. The stagnation of blood within the sinusoids of the corpora cavernosa during a physiological erection increases erythrocyte rigidity and impairs the venous outflow from the corporeal bodies. Prolongation of the erection and pain induced by ischemia of the corporeal tissues are potential consequences of these events. Most of the affected patients had repeated events that lasted for less than 3 hours and a majority of the attacks occurred during sleep, which could be due to the physiological dehydration and metabolic acidosis that accompany sleep and that increase the rigidity of erythrocytes in patients with sickle cell anemia. Priapism may be one reflection of the vasculopathy of SCD. Apart from anecdotal experiences, there is no clear evidence-based guidelines in management or medical prevention strategies. The goal of priapism treatment is to achieve detumescence and preserve erective dysfunction (Montague et al 2003). Treatment for SCD-related priapism are lacking due to the clinical misunderstanding and the ideal medical interventions have not been developed. In recent years, major scientific progress has advanced the understanding of pathophysiology especially in molecular effector pathways mediating penile erection. The medical management of recurrent or stuttering SCD-related priapism is difficult and frustrating, often involving sequential attempts with several different drugs, and clearly, more rigorous clinical research is needed in this area.

Complicated VOCs and especially priapism treatment in general and especially in SCD represent a major unmet medical need.

SEG101 (Crizanlizumab) is described in WO2008/069999, which is hereby incorporated by reference in its entirety. The present invention relates to the use of a P-selectin inhibitor, such as an anti-P-selectin antibody, such as crizanlizumab or a fragment thereof, in the treatment or prevention of a P-selectin mediated disorder, especially priapism. In particular the invention relates to a dosage regime which the inventors have found to be of a particularly good efficacy.

### BRIEF SUMMARY OF THE DISCLOSURE

Surprisingly, results from a clinical study are appropriate to provide evidence that the emergence of priapism in SCD affected patients may indeed be dependent on the vascular occlusion and that treatment thereof with inhibitors of P-selectin provides a first effective medication against priapism, especially in SCD patients and thus allows to fulfill a hitherto unmet medical need. This speaks in favor of priapism, especially priapism in SCD patients, being a P-selectin mediated disorder and/or symptom.

### BRIEF DESCRIPTION OF THE DRAWINGS

### Detailed Description

The present invention is based especially on the inventors' surprising finding that an anti-P-selectin antibody, especially when provided at a specific concentration and/or at specific time intervals, has an exceptional ability to reduce the frequency or prevent priapism in patients with SCD.

In the following, more general expressions are defined in terms of preferred more specific definitions that can replace, individually or as one or more of them, the corresponding terms in invention embodiments, thus forming more preferred invention embodiments.

In other terms: Features, integers, characteristics or compounds, described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

Treatment and/or Prevention of a P-Selectin Mediated Disorder

In the context of the present invention, the term “P-selectin mediated priapism” refers to priapism in which P-selectin plays a role, especially by forming P-selectin/PSGL-1 complexes, in the occurrence of priapism event. Often P-selectin mediated priapism is associated with increased levels of P-selectin/PSGL-1 complexes. As touched upon elsewhere in this specification, the anti-P-selectin antibody or binding fragment thereof, especially crizanlizumab, has the ability to reduce the formation of P-selectin/PSGL-1 complexes. It may also have the ability to dissociate pre-formed P-selectin/PSGL-1 complexes. Accordingly, it will be appreciated that the use of anti-P-selectin antibodies or binding fragments thereof, especially crizanlizumab or a fragment thereof, allows the (complete or at least partial) prevention of P-selectin mediated priapism by inhibiting the formation of new P-selectin/PSGL-1 complexes. It will also be appreciated that the use of anti-P-selectin antibodies or binding fragments thereof allows the treatment of existing P-selectin mediated priapism by dissociating pre-formed P-selectin/PSGL-1 complexes. Suitably, the reduction in the formation of P-selectin/PSGL-1 complexes and the dissociation of such complexes occurs during cell to cell interactions.

Priapism may be experienced by patients with sickle cell disease. The anti-P-selectin antibody or binding fragment thereof, especially crizanlizumab or a fragment thereof, may have particular utility in treating (including at least mitigating) and (completely or at least partially) preventing priapism in such patients. Suitably, the anti-P-selectin antibody or binding fragment thereof, especially crizanlizumab, may be used to treat and/or prevent sickle cell pain crisis in patients with sickle cell disease with a genotype selected from the group consisting of: HbSS, HbSC, HbSβ⁰−thalassemia and HbSβ⁰+thalassemia.

In one embodiment the patient has experienced ≥4 priapic events (unwanted erection lasting at least 60 minutes) over 26 weeks, or over 20 weeks or over 14 weeks or over 10 weeks prior to treatment.

In one embodiment the patient has at least 1 event occurring within 4 weeks prior to the first treatment.

In one embodiment, the patient may be provided with an anti-P-selectin antibody or binding fragment thereof, especially crizanlizumab or a fragment thereof, as a first line treatment for priapism, especially a P-selectin mediated priapism.

In another embodiment the patient may have received another treatment for priapism, prior to commencement of treatment in accordance with the present invention.

In another embodiment, the subject may have had at least 2, at least 3, at least 4, at least 5, at least 6, at least 7, at least 8, at least 9, or at least 10 SCPC events within the last 12 months before start of the treatment.

It shall be appreciated that there are various routes in which the subject may be provided with an anti-P-selectin antibody or binding fragment thereof. Such suitable routes may be selected from the group consisting of: intravenous, oral, parenteral, intraperitoneal, intramuscular, intravascular, intranasal, intraperitoneal, rectal, subcutaneous, transdermal and percutaneous. More suitably, the patient may be provided with an anti-P-selectin antibody or binding fragment thereof, especially crizanlizumab or a binding fragment thereof, by intravenous route. In one embodiment, the intravenous route is by injection. In another embodiment the patient is provided with anti-P-selectin antibody or binding fragment thereof, especially crizanlizumab or a binding fragment thereof, by subcutaneous route.

The anti-P-selectin antibody or binding fragment thereof, especially crizanlizumab or a binding fragment thereof, may be provided to the subject over any reasonable delivery time. Suitable delivery times may be selected from anywhere between 1 minute to 2 hours, 5 minutes to 90 minutes, 15 minutes to 70 minutes, 20 minutes to 1 hour, or 30 minutes to 50 minutes, for example. In one embodiment, the subject may be provided with an anti-P-selectin antibody or binding fragment thereof over a delivery time of 30 minutes.

Delivery times are suitably applicable to providing the anti-P-selectin antibody or binding fragment thereof by injection, preferably injection intravenously.

In one embodiment, the anti-P-selectin antibody or binding fragment thereof may be provided to the subject by intravenous injection over 30 minutes.

In an embodiment, the anti-P-selectin antibody or binding fragment thereof, especially crizanlizumab or a binding fragment thereof, may be provided to the patient in combination with a second treatment. The second treatment may be selected from any other known treatments for priapism, for example: hydroxyurea and/or L-glutamine. The combined treatment has a synergistic effect on treating the P-selectin mediated disorder.

The second treatment can also be blood transfusion or aspiration of blood and/or injection of an alpha-adrenergic agent. The second treatment can be shunt procedures allowing for increased blood flow. The second treatment could be androgen ablation therapy.

### Loading Phases and Maintenance Phases

Certain aspects of the present invention refer to the provision of an anti-P-selectin antibody or binding fragment, preferably crizanlizumab or a binding fragment thereof, to a subject, especially suffering from or expected to suffer from priapism and in particular being in need of such treatment, in a loading phase, followed by further provision of the antibody or binding fragment, preferably crizanlizumab or a binding fragment thereof, in a maintenance phase. In such an embodiment, the subject receives a first amount of the antibody or binding fragment over a given period of time during the loading phase, and then receives a lower amount of the antibody or binding fragment over a given period of time, suitably the same given period of time, during the maintenance phase. The different amounts of the antibody required by the loading phase and the maintenance phase may be provided by providing different doses of the antibody and/or by employing different intervals of time between administrations of the antibody. For example, during the maintenance phase the antibody may be provided at essentially the same dose as used during the loading phase, but with longer intervals of time between each incidence of administration. Alternatively, the time between intervals of administration may be the same in each of the loading and maintenance phases, but the dose of antibody provided in each incidence of administration during the maintenance phase may be lower.

Merely by way of example, a suitable loading phase may involve the provision to the subject of a suitable antibody, or binding fragment thereof, in an amount of approximately more than 5 mg/kg, e.g. 10 mg/kg per week of the loading phase (whether this is provided weekly, bi-weekly, or otherwise). In such an embodiment of the invention a suitable maintenance phase may involve the provision to the subject of the antibody, or binding fragment thereof, in an amount of approximately 5 mg/kg per week of the maintenance phase (for example by provision bi-weekly or especially every four weeks). Generally the amount of the antibody, or binding fragment thereof, provided per week of a loading phase may be approximately double that provided per week of the maintenance phase.

As another example, a suitable loading phase may involve the provision to the subject of a suitable antibody, or binding fragment thereof, in an amount of approximately 7.5 or preferably 5 mg/kg bi-weekly, or otherwise. In such an embodiment of the invention a suitable maintenance phase may involve the provision to the subject of the antibody, or binding fragment thereof, in an amount of approximately 7.5 or preferably 5 mg/kg per week of the maintenance phase (for example by provision bi-weekly or especially every four weeks). Generally the amount of the antibody, or binding fragment thereof, provided per week of a loading phase may be approximately double that provided on a per week basis of the maintenance phase.

It will be appreciated that, since the length of the maintenance phase may be much longer than the loading phase, the total amount of the antibody or binding fragment received by the subject over the maintenance phase may be much more than that provided during the relatively shorter loading phase. However, the amount of the antibody that the subject will receive over a set period of the maintenance phase will be lower than the amount that would be received over the same period of the loading phase.

The loading and maintenance phases required by such embodiments of the invention may be put into practice by use of the loading and maintenance doses, and associated administration regimens, considered below.

Various aspects of the invention refer to the average time intervals between maintenance doses, and to average time intervals following the one or more loading doses. It will be recognised that suitable average time intervals may be achieved by varying the number of doses, and the individual intervals between maintenance doses, or following loading doses (whether the loading dose in question is followed by a further loading dose, or a maintenance dose). The following paragraphs provide examples of suitable individual time intervals that may be used in achieving a desired average time interval.

Where +/−3 days is mentioned this may be replaced with +/−2 days, +/−1 day or +/−0 days.

### Determining Effectiveness

In another aspect, the inventors have surprisingly found that providing anti-P-selectin antibodies or binding fragments, preferably crizanlizumab or a binding fragment thereof, to patients with P-selectin mediated disorders may lower the levels of soluble P-selectin in a sample from the patient. The inventors believe that this finding may be of utility in determining and/or monitoring the effectiveness of an anti-P-selectin antibody or binding fragment thereof treatment in a subject with a P-selectin mediated disorder and/or symptoms, especially priapism.

Accordingly, the invention also relates to a method of determining effectiveness of treatment with an anti-P-selectin antibody or binding fragment thereof, preferably crizanlizumab or a binding fragment thereof, the method comprising the steps of:
measuring levels of soluble P-selectin in a sample from a subject provided with an anti-P-selectin antibody or binding fragment thereof, preferably crizanlizumab or a binding fragment thereof, and
comparing the subject's soluble P-selectin levels to a reference value, and
thereby determining the effectiveness of the treatment.

In one embodiment, the method is for determining effectiveness of treatment of priapism with crizanlizumab or a binding fragment thereof in a patient. In one further embodiment said patient has sickle cell disease.

Throughout the description and claims of this specification, the singular encompasses the plural unless the context otherwise requires. In particular, where the indefinite article is used, the specification is to be understood as contemplating plurality as well as singularity, unless the context requires otherwise.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The reader's attention is directed to all papers and documents which are filed concurrently with or previous to this specification in connection with this application and which are open to public inspection with this specification, and the contents of all such papers and documents are incorporated herein by reference.

In one aspect the present invention provides an anti-P-selectin antibody or binding fragment thereof, crizanlizumab or a binding fragment thereof, for use in the treatment or prevention of (especially P-selectin mediated) priapism in a patient, wherein the first two doses of said antibody or binding fragment thereof is provided 2 weeks (+/−3 days) apart followed by further doses provided every 4 weeks (+/−3 days), wherein each dose is between 2.5 mg per kg body weight (2.5 mg/kg) to 20 mg/kg, preferably 2.5 mg/kg to 10 mg/kg, preferably 2.5 mg/kg to 7.5 mg/kg and preferably wherein the interval between the last loading dose and the first maintenance dose is 4 weeks (+/−3 days). In one preferred embodiment, the loading dose is 7.5 mg7 kg or in particular 5 mg/kg, the maintenance dose is 7.5 mg/kg or in particular 5 mg/kg, and the time interval between the last loading and first maintenance dose is 4 weeks (+/−3 days).

In another aspect, the present invention provides a method of reducing the frequency of priapism events comprising administrating an therapeutically effective amount of an anti-P-selectin antibody or binding fragment thereof, especially crizanlizumab or a binding fragment thereof, to a subject in need thereof, wherein the first two doses of said antibody or binding fragment thereof is provided 2 weeks (+/−3 days) apart followed by further doses provided every 4 weeks (+/−3 days), wherein each dose is between 2.5 mg/kg to 20 mg/kg, or between 2.5 mg/kg to 10 mg/kg, or between 2.5 mg/kg to 7.5 mg/kg.

In one embodiment, each of said doses is 2.5 mg/kg. In another particular embodiment, each of said doses is 5 mg/kg. In another embodiment, each of said doses is 7.5 mg/kg. When the dose is initially 7.5 mg/kg, the dose is allowed to be reduced for safety reasons to 5 mg/kg at any time after the loading dose, normally 1 month, 2 months, 3 months, 4 months, 5 months, 6 months, 7 months, 8 months, 9 months, 10 months, or 11 months after the last loading dose. Safety parameters are monitored by health care professionals during the treatment.

The term “anti-P-selectin antibody or binding fragment thereof” as used herein refers to an antibody, or binding fragment thereof, which comprises a P-selectin binding domain. The binding of the antibody (or binding fragment thereof) to P-selectin inhibits the binding of P-selectin to PSGL-1 and thereby reduces the formation of P-selectin/PSGL-1 complexes. Suitably, the anti-P-selectin antibody or binding fragment thereof may reduce the formation of P-selectin/PSGL-1 complexes by at least 30%, at least 40%, at least 50%, at least 60%, at least 70%, at least 80%, at least 90%, at least 95%, at least 96%, at least 97%, at least 98%, at least 99%, or more as compared to a suitable control (for example a sample without the presence of an anti-P-selectin antibody or binding fragment thereof).

Additionally or alternatively, an anti-P-selectin antibody or binding thereof may dissociate preformed P-selectin/PSGL-1 complexes. In a suitable embodiment antibody or binding fragment thereof may dissociate at least 30%, at least 40%, at least 50%, at least 60%, at least 70%, at least 80%, at least 90%, at least 95%, at least 96%, at least 97%, at least 98%, at least 99%, or more of preformed P-selectin/PSGL-1 complexes. As before, this property may be compared to a suitable control (for example a sample without the presence of an anti-P-selectin antibody or binding fragment thereof).

Additionally or alternatively, an anti-P-selectin antibody or binding thereof may refer to an antibody or binding thereof that is capable of binding to P-selectin specifically, i.e. it binds to P-selectin with an affinity higher than an antibody that is well known not to bind P-selectin specifically. The affinity can be suitably determined by, for example, surface plasmon resonance (BIAcore™) assay. Ideally, the K_{d} of a P-selectin antibody or a fragment thereof is ≤1000 nM, or ≤500 nM, or ≤100 nM, or ≤50 nM, or more preferably by a K_{d}≤25 nM, and still more preferably by a K_{d}≤10 nM, and even more preferably by a K_{d}≤5 nM, or ≤1 nM, or ≤0.1 nM.

In one embodiment, the anti-P-selectin antibody or a binding fragment thereof is crizanlizumab or a binding fragment thereof.

In one embodiment, the anti-P-selectin antibody or (P-selectin) binding fragment thereof may bind P-selectin at any suitable epitope. Suitably, the anti-P-selectin antibody or binding fragment thereof may bind an epitope which is found in the P-selectin lectin-like domain.

In one embodiment, the anti-P-selectin antibody of binding fragment thereof binds P-selectin at amino acid positions 1 to 35 of SEQ ID NO: 1. Suitably the anti-P-selectin antibody or binding fragment thereof binds P-selectin at amino acid positions 4 to 23 of SEQ ID NO: 1. More suitably, the anti-P-selectin antibody or binding fragment thereof binds P-selectin at amino acid positions 4, 14, 17, 21, and 22 of SEQ ID NO: 1.

In one embodiment, the anti-P-selectin antibody or binding fragment thereof comprises a light chain variable region having a CDR sequence selected from the group consisting of KASQSVDYDGHSYMN (SEQ ID NO: 2), AASNLES (SEQ ID NO: 3) and QQSDENPLT (SEQ ID NO: 4).

Specific features of the humanised antibody SelG1, which is a suitable antibody to be employed in the methods and medical uses of the present invention, are set out below.

In a suitable embodiment, the anti-P-selectin antibody or binding fragment thereof may comprise a light chain variable CDR with an amino acid sequence that varies from a sequence selected from the group consisting of KASQSVDYDGHSYMN (SEQ ID NO: 2), AASNLES (SEQ ID NO: 3) and QQSDENPLT (SEQ ID NO: 4) by no more than four amino acid residues, by no more than three amino acid residues, by no more than two amino acid residues, or by no more than one amino acid residue.

In one embodiment the anti-P-selectin antibody or binding fragment thereof comprises a light chain variable region comprising SEQ ID NO: 5. Suitably, the anti-P-selectin antibody or binding fragment thereof comprises of a light chain variable region consisting of SEQ ID NO: 5.

In a suitable embodiment the anti-P-selectin antibody or binding fragment thereof comprises a light chain variable region which comprises or consists of a polypeptide which is at least 90%, at least 95%, at least 96%, at least 97%, at least 98%, or at least 99% identical to SEQ ID NO: 5.

In one embodiment, the anti-P-selectin antibody or binding fragment thereof comprises a heavy chain variable region having a CDR sequence selected from the group consisting of SYDIN (SEQ ID NO: 6), WIYPGDGSIKYNEKFKG (SEQ ID NO: 7) and RGEYGNYEGAMDY (SEQ ID NO: 8).

In a suitable embodiment, the anti-P-selectin antibody or binding fragment thereof may comprise a heavy chain variable CDR with an amino acid sequence that varies from a sequence selected from the group consisting of SYDIN (SEQ ID NO: 6), WIYPGDGSIKYNEKFKG (SEQ ID NO: 7) and RGEYGNYEGAMDY (SEQ ID NO: 8) by no more than four amino acid residues, by no more than three amino acid residues, by no more than two amino acid residues, or by no more than one amino acid residue.

In one embodiment the anti-P-selectin antibody or binding fragment thereof comprises a heavy chain variable region comprising SEQ ID NO: 9. Suitably, the anti-P-selectin antibody or binding fragment thereof comprises a heavy chain variable region consisting of SEQ ID NO: 9.

In a suitable embodiment the anti-P-selectin antibody or binding fragment thereof comprises a heavy chain variable region which comprises or consists of a polypeptide which is at least 90%, at least 95%, at least 96%, at least 97%, at least 98%, or at least 99% identical to SEQ ID NO: 9.

In one embodiment the anti-P-selectin antibody or binding fragment thereof comprises a heavy chain variable region comprising three CDRs comprising, consisting essentially of or consisting of SEQ ID NO: 6, SEQ ID NO: 7, and SEQ ID NO: 8, respectively and a light chain variable region comprising three CDRs comprising, consisting essentially of or consisting of SEQ ID NO: 2, SEQ ID NO: 3, and SEQ ID NO: 4, respectively.

In one embodiment the anti-P-selectin antibody or binding fragment thereof comprises a light chain variable region comprising, consisting essentially of or consisting of the sequence SEQ ID NO: 5 and a heavy chain variable region comprising, consisting essentially of or consisting of the sequence SEQ ID NO: 9.

In a suitable embodiment, the antibody or binding fragment thereof may further comprise a constant region. The constant region may comprise a light chain constant region and/or a heavy chain constant region.

The light chain constant region may comprise a human kappa chain or a human lambda chain. Alternatively, the light chain constant region may consist of a human kappa chain or consist of a human lambda chain. Suitably the human kappa chain may be according to SEQ ID NO: 10. Alternatively the human kappa chain may be at least 90%, at least 95%, at least 96%, at least 97%, at least 98%, or at least 99% identical to SEQ ID NO: 10.

The heavy chain constant region may be selected from the group consisting of: IgG, IgA, IgD, IgE, and IgM. Immunoglobulin constant regions may be further classified into isotypes. Thus, the heavy chain constant region may be selected from the group consisting of: IgG₂, IgG₁ IgG₃ and IgG₄.

In one embodiment the heavy chain constant region may comprise an IgG. More suitably, the heavy chain constant region may comprise an IgG₂.

Alternatively the heavy chain constant region may consist of an IgG. More suitably, the heavy chain of the constant region may consist of an IgG₂. Suitably the IgG₂ may be according to SEQ ID NO: 11. Alternatively the IgG₂ may be at least 90%, at least 95%, at least 96%, at least 97%, at least 98%, or at least 99% identical to SEQ ID NO: 11. For example, an IgG₂ sequence to be employed in the invention may comprise five or less, four or less, three or less, two or less, or one or less mutations in IgG₂ sequence according to SEQ ID NO: 11. Suitably, the IgG₂ sequence to be employed in the invention may comprise one mutation in the sequence according to SEQ ID NO: 11. In such an embodiment, the IgG₂ to be employed in the invention suitably has a sequence according to SEQ ID NO: 23. An IgG₂ according to SEQ ID NO: 23 may be desirable in order to further reduce complement activation.

In one embodiment the anti-P-selectin antibody comprises a light chain which is at least 90%, at least 95%, at least 96%, at least 97%, at least 98%, at least 99% identical to SEQ ID NO: 12. Suitably, the anti-P-selectin antibody comprises a light chain according to SEQ ID NO: 12.

In one embodiment the anti-P-selectin antibody comprises a heavy chain which is at least 90%, at least 95%, at least 96%, at least 97%, at least 98%, at least 99% identical to SEQ ID NO: 13. Suitably, the anti-P-selectin antibody comprises a heavy chain according to SEQ ID NO: 13.

In a suitable embodiment the anti-P-selectin antibody comprises a light chain which is at least 90%, at least 95%, at least 96%, at least 97%, at least 98%, at least 99% identical to SEQ ID NO: 12, and a heavy chain which is at least 90%, at least 95%, at least 96%, at least 97%, at least 98%, at least 99% identical to SEQ ID NO: 13. Suitably the anti-P-selectin antibody comprises a light chain according to SEQ ID NO: 12, and a heavy chain according to SEQ ID NO: 13.

Other suitable anti-P-selectin antibodies are disclosed in WO2005/100402, WO1993/021956 and WO1994/025067, which are hereby incorporated by reference in their entirety. In one embodiment, the suitable anti-P-selectin antibody or a fragment thereof is inclacumab or a binding fragment thereof.

In the context of the present invention, the term “binding fragment” as used herein refers to a portion of an antibody capable of binding a P-selectin epitope.

In one embodiment, the binding fragment may comprise an antigen binding and/or variable region. Merely by way of example, a suitable binding fragment may be selected from the group consisting of Fab, Fab′, F(ab′)2, Fv and scFv. Suitable binding fragments may be produced by various methods known in the art. A Fab′ fragment, for example, may be produced by papain digestion of an antibody. A F(ab′)2 fragment, for example, may be produced by pepsin digestion of an antibody.

In one embodiment, the anti-P-selectin antibody or binding fragment thereof, preferably crizalizumab or a binding fragment thereof, has very low immunogenicity. More suitably, the anti-P-selectin antibody, preferably Crizanlizumab, has no or low immunogenicity. The term immunogenicity as used herein, refers to the ability of the antibody or binding fragment thereof to trigger the production of neutralising antibodies against it in the subject.

As mentioned elsewhere in this specification, the generation of neutralising antibodies is highly undesirable, as they may neutralise a therapeutic antibody (or binding fragment thereof), rendering it ineffective. The production of neutralising antibodies may result in a decrease in the levels of the therapeutic antibodies in the subject. Thus, it will be appreciated that a consistent level or amount of therapeutic antibodies in the subject (for example in a serum sample from the subject) may be indicative that no such neutralising antibodies have been produced, and thus that the therapeutic antibody has little or no immunogenicity. By the term consistent it is meant that the level of the therapeutic antibody does not fluctuate in a subject by more than 5%, more than 10%, more that 15%, more than 20%, more than 25%, more than 30%, more than 35%, more than 45%, or more than 50% during the maintenance phase.

### PARTICULAR EMBODIMENTS OF THE PRESENT INVENTION

### Embodiments (a)

1a. An anti-P-selectin antibody or binding fragment thereof, for use in the treatment or prevention of or reducing the frequency of a P-selectin mediated disorder, especially priapism, wherein the antibody or fragment thereof is administered to a patient or subject suffering from priapism.

2a. An anti-P-selectin antibody or binding fragment thereof for use according to embodiment 1a, wherein the patient or subject is suffering from Sickle Cell Disease (SCD).

3a. An anti-P-selectin antibody or binding fragment thereof for use according to embodiment 1a or 2a, wherein the anti-P-selectin antibody is crizanlizumab or a fragment P-selectin binding thereof.

4a. An anti-P-selectin antibody or binding fragment thereof for use according to any one of embodiments 1a to 3a, wherein the patient or subject receives co-treatment with another drug, especially hydroxyurea, hydroxycarbamide, L-glutamine, Erythropoietin-stimulating agents, acetylsalicylic acid, nonsteroidal anti-inflammatory drug, or anticoagulants at prophylactic doses, or any combination of two or more thereof.

5a. An anti-P-selectin antibody or binding fragment thereof for use according to any one of embodiments 1a to 3a, as monotherapy, that is, without any other drug used in treatment of SCD or especially VOC.

6a. An anti-P-selectin antibody or binding fragment thereof for use according to any one of embodiments 1a to 3a or 5a as monotherapy with crizanlizumab.

7a. An anti-P-selectin antibody or binding fragment thereof for use according to any one of embodiments 1a to 6a, wherein the antibody or fragment thereof, especially crizanlizumab is administered in a loading dose, preferably two loading doses, followed by a maintenance dose.

8a. An anti-P-selectin antibody or binding fragment thereof for use according to embodiment 7a, wherein the loading dose is 5 mg/kg or 7.5 mg/kg and is administered twice in a time interval of 2 weeks+/−3 days, and the maintenance dosage is then administered 4 weeks+/−3 days after the second loading dose administration and then at regular intervals of 4 weeks+/−3 days.

9a. An anti-P-selectin antibody or binding fragment thereof for use according to any one of embodiments 1a to 8a, where the priapism treated is defined as an unwanted or painful penile erection lasting at least 60 minutes.

10a. An anti-P-selectin antibody or binding fragment thereof for use according to any one of embodiments 1a to 9a, where the anti-P-Selectin antibody or binding fragment thereof comprises a heavy chain variable region comprising three CDRs comprising, consisting essentially of or consisting of SEQ ID NO: 6, SEQ ID NO: 7, and SEQ ID NO: 8, respectively and a light chain variable region comprising three CDRs comprising, consisting essentially of or consisting of SEQ ID NO: 2, SEQ ID NO: 3, and SEQ ID NO: 4, respectively.

11a. An anti-P-selectin antibody or binding fragment thereof for use according to any one of embodiments 1a to 10a, wherein the anti-P-selectin antibody or binding fragment thereof is provided to the subject by an intravenous route, especially by infusion.

12a. An anti-P-selectin antibody or binding fragment thereof, for use according to any one of embodiments 1a to 11a, wherein the antibody or binding fragment thereof binds P-selectin at an epitope corresponding to amino acids 1 to 35 of SEQ ID NO: 1.

13a. An anti-P-selectin antibody or binding fragment thereof, for use according to any one of embodiments 1a to 12a, wherein the antibody or binding fragment thereof comprises a heavy chain sequence which is at least 90% identical to SEQ ID NO: 13.

14a. An anti-P-selectin antibody or binding fragment thereof, for use according to any one of embodiments 1a to 13a, wherein the antibody or binding fragment thereof comprises a light chain sequence which is at least 90% identical to SEQ ID NO: 12.

15a. An anti-P-selectin antibody or binding fragment thereof, for use according to any one of embodiments 1a to 14a, wherein the antibody comprises a heavy chain sequence which is at least 90% identical to SEQ ID NO: 13 and a light chain sequence which is at least 90% identical to SEQ ID NO: 12.

### Embodiments (b)

1b. A method of treating priapism in a patient or subject, especially in need of such treatment, comprising administering a—preferably pharmaceutically effective—amount of an anti-P-selectin antibody or binding fragment thereof to said patient or subject, especially suffering from or expected to suffer from priapism.

2b. The method according to embodiment 1b, wherein the patient or subject is suffering from Sickle Cell Disease (SCD).

3b. The method according to embodiment 1b or 2b, wherein the anti-P-selectin antibody is crizanlizumab or a fragment P-selectin binding thereof.

4b. The method according to any one of embodiments 1 b to 3b, wherein the patient or subject receives co-treatment with another drug, especially hydroxyurea, hydroxycarbamide, L-glutamine, Erythropoietin-stimulating agents, acetylsalicylic acid, nonsteroidal anti-inflammatory drug, or anticoagulants at prophylactic doses, or any combination of two or more thereof.

5b. The method according to any one of embodiments 1 b to 3b as monotherapy, that is, without any other drug used in treatment of SCD or especially VOC.

6b. The method according to any one of embodiments 1b to 3b or 5b as monotherapy with crizanlizumab.

7b. The method according to any one of embodiments 1b to 6b, wherein the antibody or fragment thereof, especially crizanlizumab is administered in a loading dose, preferably two loading doses, followed by a maintenance dose.

8b. The method according to embodiment 7b, wherein the loading dose is 5 mg/kg or 7.5 mg/kg and is administered twice in a time interval of 2 weeks+/−3 days, and the maintenance dosage is then administered 4 weeks+/−3 days after the second loading dose administration and then at regular intervals of 4 weeks+/−3 days.

9b. The method according to any one of embodiments 1a to 8b, where the priapism treated is defined as an unwanted or painful penile erection lasting at least 60 minutes.

10b. The method according to any one of embodiments 1 b to 9b, where the anti-P-Selectin antibody or binding fragment thereof comprises a heavy chain variable region comprising three CDRs comprising, consisting essentially of or consisting of SEQ ID NO: 6, SEQ ID NO: 7, and SEQ ID NO: 8, respectively and a light chain variable region comprising three CDRs comprising, consisting essentially of or consisting of SEQ ID NO: 2, SEQ ID NO: 3, and SEQ ID NO: 4, respectively.

11b. The method according to any one of embodiments 1b to 10b, wherein the anti-P-selectin antibody or binding fragment thereof is provided to the subject by an intravenous route, especially by infusion.

12b. The method according to any one of embodiments 1b to 11b, wherein the antibody or binding fragment thereof binds P-selectin at an epitope corresponding to amino acids 1 to 35 of SEQ ID NO: 1.

13b. The method according to any one of embodiments 1b to 12b, wherein the antibody or binding fragment thereof comprises a heavy chain sequence which is at least 90% identical to SEQ ID NO: 13.

14b. The method according to any one of embodiments 1b to 13b, wherein the antibody or binding fragment thereof comprises a light chain sequence which is at least 90% identical to SEQ ID NO: 12.

15b. The method according to any one of embodiments 1 b to 14b, wherein the antibody comprises a heavy chain sequence which is at least 90% identical to SEQ ID NO: 13 and a light chain sequence which is at least 90% identical to SEQ ID NO: 12.

### Embodiments (c)

1c. The use of an anti-P-selectin antibody or binding fragment thereof in the preparation of a medicament for the treatment or prevention of a P-selectin mediated disorder, especially priapism, wherein the antibody or fragment thereof is administered to a patient or subject suffering from priapism.

2c. The use according to embodiment 1c, wherein the patient or subject is suffering from Sickle Cell Disease (SCD).

3c. The use according to embodiment 1c or 2c, wherein the anti-P-selectin antibody is crizanlizumab or a fragment P-selectin binding thereof.

4c. The use according to any one of embodiments 1c to 3c, wherein the patient or subject receives co-treatment with another drug, especially hydroxyurea, hydroxycarbamide, L-glutamine, Erythropoietin-stimulating agents, acetylsalicylic acid, nonsteroidal anti-inflammatory drug, or anticoagulants at prophylactic doses, or any combination of two or more thereof.

5c. The use according to any one of embodiments 1c to 3c, in monotherapy, that is, without any other drug used in treatment of SCD or especially VOC.

6c. The use according to any one of embodiments 1c to 3c or 5c in monotherapy with crizanlizumab.

7c. The use according to any one of embodiments 1c to 6c, wherein the antibody or fragment thereof, especially crizanlizumab, is administered in a loading dose, preferably two loading doses, followed by a maintenance dose.

8c. The use according to embodiment 7c, wherein the loading dose is 5 mg/kg or 7.5 mg/kg and is administered twice in a time interval of 2 weeks+/−3 days, and the maintenance dosage is then administered 4 weeks+/−3 days after the second loading dose administration and then at regular intervals of 4 weeks+/−3 days.

9c. The use according to any one of embodiments 1c to 8c, where the priapism treated is defined as an unwanted or painful penile erection lasting at least 60 minutes.

10c. The use according to any one of embodiments 1c to 9c, where the anti-P-Selectin antibody or binding fragment thereof comprises a heavy chain variable region comprising three CDRs comprising, consisting essentially of or consisting of SEQ ID NO: 6, SEQ ID NO: 7, and SEQ ID NO: 8, respectively and a light chain variable region comprising three CDRs comprising, consisting essentially of or consisting of SEQ ID NO: 2, SEQ ID NO: 3, and SEQ ID NO: 4, respectively.

11c. The use according to any one of embodiments 1c to 10c, wherein the anti-P-selectin antibody or binding fragment thereof is provided to the subject by an intravenous route, especially by infusion.

12c. The use according to any one of embodiments 1c to 11c, wherein the antibody or binding fragment thereof binds P-selectin at an epitope corresponding to amino acids 1 to 35 of SEQ ID NO: 1.

13c. The use according to any one of embodiments 1c to 12c, wherein the antibody or binding fragment thereof comprises a heavy chain sequence which is at least 90% identical to SEQ ID NO: 13.

14c. The use according to any one of embodiments 1c to 13c, wherein the antibody or binding fragment thereof comprises a light chain sequence which is at least 90% identical to SEQ ID NO: 12.

15c. The use according to any one of embodiments 1c to 14c, wherein the antibody comprises a heavy chain sequence which is at least 90% identical to SEQ ID NO: 13 and a light chain sequence which is at least 90% identical to SEQ ID NO: 12.

### Embodiments (d)

1d. A pharmaceutical preparation, especially for infusion, comprising, beyond an optional pharmaceutically acceptable carrier material, an anti-P-selectin antibody or binding fragment thereof for the treatment or prevention of a P-selectin mediated disorder, especially priapism, wherein the antibody or fragment thereof is administered to a patient or subject suffering from priapism.

2d. The pharmaceutical preparation for treatment according to embodiment 1d, wherein the patient or subject is suffering from Sickle Cell Disease (SCD).

3d. The pharmaceutical preparation for treatment according to embodiment 1d or 2d, wherein the anti-P-selectin antibody is crizanlizumab or a fragment P-selectin binding thereof.

4d. The pharmaceutical preparation for treatment according to any one of embodiments 1d to 3d, wherein the patient or subject receives co-treatment with another drug, especially hydroxyurea, hydroxycarbamide, L-glutamine, Erythropoietin-stimulating agents, acetylsalicylic acid, nonsteroidal anti-inflammatory drug, or anticoagulants at prophylactic doses, or any combination of two or more thereof.

5d. The pharmaceutical preparation for treatment according to any one of embodiments 1d to 3d, in monotherapy, that is, without any other drug used in treatment of SCD or especially VOC.

6d. The pharmaceutical preparation for treatment according to any one of embodiments 1d to 3d or 5d in monotherapy with crizanlizumab.

7d. The pharmaceutical preparation for treatment according to any one of embodiments 1d to 6d, wherein the antibody or fragment thereof, especially crizanlizumab, is administered in a loading dose, preferably two loading doses, followed by a maintenance dose.

8d. The pharmaceutical preparation for treatment according to embodiment 7d, wherein the loading dose is 5 mg/kg or 7.5 mg/kg and is administered twice in a time interval of 2 weeks+/−3 days, and the maintenance dosage is then administered 4 weeks+/−3 days after the second loading dose administration and then at regular intervals of 4 weeks+/−3 days.

9d. The pharmaceutical preparation for treatment according to any one of embodiments 1d to 8d, where the priapism treated is defined as an unwanted or painful penile erection lasting at least 60 minutes.

10d. The pharmaceutical preparation for treatment according to any one of embodiments 1d to 9d, where the anti-P-Selectin antibody or binding fragment thereof comprises a heavy chain variable region comprising three CDRs comprising, consisting essentially of or consisting of SEQ ID NO: 6, SEQ ID NO: 7, and SEQ ID NO: 8, respectively and a light chain variable region comprising three CDRs comprising, consisting essentially of or consisting of SEQ ID NO: 2, SEQ ID NO: 3, and SEQ ID NO: 4, respectively.

11d. The pharmaceutical preparation for treatment according to any one of embodiments 1c to 10c, wherein the anti-P-selectin antibody or binding fragment thereof is provided to the subject by an intravenous route, especially by infusion.

12d. The pharmaceutical preparation for treatment according to any one of embodiments 1d to 11d, wherein the antibody or binding fragment thereof binds P-selectin at an epitope corresponding to amino acids 1 to 35 of SEQ ID NO: 1.

13d. The pharmaceutical preparation for treatment according to any one of embodiments 1d to 12d, wherein the antibody or binding fragment thereof comprises a heavy chain sequence which is at least 90% identical to SEQ ID NO: 13.

14d. The pharmaceutical preparation for treatment according to any one of embodiments 1d to 13d, wherein the antibody or binding fragment thereof comprises a light chain sequence which is at least 90% identical to SEQ ID NO: 12.

15d. The pharmaceutical preparation for treatment according to any one of embodiments 1d to 14d, wherein the antibody comprises a heavy chain sequence which is at least 90% identical to SEQ ID NO: 13 and a light chain sequence which is at least 90% identical to SEQ ID NO: 12.

Preferably, in all embodiments presented above and below the preservation of sexual function in patients affected with priapism is a goal.

Preferably, in all embodiments presented above and below the patient suffering from priapism has a priapism episode once a month or more often.

### EXAMPLES

### Example 1: Treatment of a Patient Suffering from Priapism with Crizanlizumab

In the SUSTAIN (placebo controlled Phase 2) trial (Ataga K I, Kutlar A, Kanter J, et al. Crizanlizumab for the prevention of pain crises in sickle cell disease. N Engl J Med 2017; 376:429-39. DOI: 10.1056/NEJMoa1611770) Treatment of SCD patients with crizanlizumab at 5.0 mg/kg demonstrated statistically significant, clinically meaningful efficacy results versus placebo in reduction of annual rate of VOCs leading to healthcare visit and delay in onset of VOCs.

Following completion of the SUSTAIN trial, one subject stayed presented frequent priapism about three times a week. After a formal approval, the subject was re-initiated with crizanlizumab seven months after his last treatment in the SUSTAIN trial. By May 17, 2019, the patient has maintained total control of crisis and disappearance of the occurrence of priapism and pain crisis since re-initiation of crizanlizumab. To this date (June 2019) this subject continues to receive crizanlizumab.

The subject has been on the medication till now (May 2020) and had no crisis during the entire period.

### Example 2: A Prospective Phase II, Open-Label, Single-Arm, Multicenter, Study to Assess Efficacy and Safety of SEG101 (Crizanlizumab), in Sickle Cell Disease Patients with Priapism (SPARTAN)

Preclinical studies in transgenic mice have shown that SCD promotes an increased P-selectin expression in several vascular beds of various organs (e.g. lung, heart, small bowel, large bowel, penis etc.). The penis is the only organ where the vascular bed exhibited an increased expression of both P- and E-selectin, which may be relevant to a SCD-related priapism. Although, priapism has been attributed to both ischemic and non-ischemic causes, vaso-occlusion-induced ischemia is now generally thought to account for the priapism associated with SCD.

The purpose of this study is to evaluate the effect of crizanlizumab on priapic events in sickle cell disease patients with a history of priapism. We collect data for 26 weeks prior to the treatment phase to create a baseline for priapic events (defined as an event of an unwanted painful erection lasting greater than 60 minutes), acute priapic events (which is defined as an event of an unwanted, painful erection lasting more than 4 hours), uncomplicated and complicated vaso-occlusive crisis (VOC), based on the hypothesis is crizanlizumab treatment reduces priapic events by at least 25% in SCD patients with priapism.

The Primary Objective is to evaluate the clinical efficacy (percent reduction from baseline) of crizanlizumab in SCD-related priapism.

Among the Secondary Objectives, the following are aimed at:
To evaluate the clinical efficacy (rate of priapic events) of crizanlizumab in SCD-related priapism
To evaluate the clinical efficacy of crizanlizumab in SCD-related acute priapism
To evaluate the clinical efficacy of crizanlizumab for uncomplicated VOC (defined as an acute event of pain with no known cause for pain other than a vaso-occlusive event; and requiring treatment with a parenteral or oral opioids or other parenteral analgesic; but is NOT classified as an acute chest syndrome, hepatic sequestration, splenic sequestration or priapism and these events are not adjudicated).
To evaluate the clinical efficacy of crizanlizumab in complicated VOCs (acute chest syndrome, hepatic sequestration, splenic sequestration, and especially acute priapism)
To further assess the safety and tolerability of crizanlizumab

Among the Exploratory Objectives, the following are examined:
To evaluate the clinical efficacy of crizanlizumab on leg ulcers (improve the severity indices for the leg ulcer based on depth by 26 and 52 weeks)
To evaluate quality of life (measures of Quality of Life via a Priapism Impact Profile (PIP) tool
Measure of healthcare resource utilization due to SCD events (e.g. number of ER visits, frequency and duration of hospitalization etc.) by 26 weeks and 52 weeks.
To evaluate the clinical efficacy of crizanlizumab on penile fibrosis based on USG (UltrasonogrPHY) by 52 weeks.

### Study Design

This is a multicenter, prospective, phase II, single-arm, open-label study to assess the efficacy and safety of crizanlizumab in SCD patients with priapism. A total of approximately 56 male subjects aged ≥16 years, who meet all of the inclusion criteria and none of the exclusion criteria are included in the study.

The baseline period is defined as 26 weeks consisting of 14 weeks pre-screening and 12 weeks screening. Eligible subjects are treated with crizanlizumab at a dose of 5.0 mg/kg. From the first screening visit until the end of follow-up, the total study duration is 79 weeks (including 12 weeks of screening, 52 weeks of treatment and 15 weeks of follow-up period). The primary analysis of the study is conducted by 26 weeks to assess efficacy of crizanlizumab in this patient population. The secondary and exploratory endpoints are assessed by 26 weeks and/or 52 weeks. Subjects are followed in the mandatory safety follow-up period until 105 days (15 weeks) after the last dosing.

### Pre-Screening

Before participating in the study, information to determine key eligibility criteria is collected as a part of priapism medical history questionnaire.

### Screening Phase

Eighty-four days (12 weeks) before start of treatment, written informed consent, according to local guidelines, is signed by the subjects and prior to any study related screening procedures are performed. During this period, subjects have 3 visits and be monitored for priapic events to determine their eligibility to the trial using an electronic reporting system. All screening evaluations must be performed during the screening period from Day −84 to Day −1.

At the first screening visit after signing the ICF (at Day 84), subjects are briefed about the study requirements, including the instruction on how to use the electronic device(s) required to record the priapic event(s) information. At the first screening visit, the prospective subjects are also asked about their prior medical history related to priapism. This information is collected via a priapism medical history questionnaire.

### Treatment Phase

Once eligibility criteria have been confirmed by Novartis via the eligibility checklist, the subject receives investigational treatment. Subjects receive investigational treatment by IV infusion over 30 min on Week 1 Day 1, Week 3 Day 1, and then on Day 1 of every 4-week cycle.

Safety is monitored as below. Subjects receive investigational treatment for a maximum of 1 year (52 weeks) or until unacceptable toxicity, death, lost to follow-up or discontinued from the study treatment for any other reasons prior to 52 weeks. Following the treatment discontinuation, subjects perform an End of Treatment visit.

### Population

A total of 56 male patients with SCD-related priapism aged ≥16 years is recruited across the United States. To be considered for this study, patients must have had ≥4 priapic events during the 14 weeks prior to screening and having at least 3 priapic events during the 12 week screening period with at least 1 event occurring within 4 weeks prior to the first treatment.

### Key Inclusion Criteria

Subjects eligible for inclusion in this study must meet all of the following criteria:

1. Signed informed consent and applicable adolescent assent and/or parental consent for adolescent subjects, obligatorily obtained prior to participation in the study

2. Male patients aged 16 years and above

3. Confirmed diagnosis of SCD by hemoglobin electrophoresis or high performance liquid chromatography. All SCD genotypes are eligible (HbSS, HbSβ0, HbSC, HbSβ+, and others)

4. Patients who have experienced ≥4 priapic events (unwanted erection lasting at least 60 minutes) over 14 week pre-screening

5. Patients who have experienced at least 3 priapic events (unwanted erection lasting at least 60 minutes) during the 12 week screening period with at least 1 event occurring within 4 weeks prior to the first treatment.

6. If receiving hydroxyurea/hydroxycarbamide or L-glutamine or erythropoietin stimulating agent, must have been receiving the drug for at least 14 weeks prior to screening and plan to continue taking the drug at the same dose and schedule during the trial

7. If receiving prophylactic treatment for priapism, must have been receiving the drug for at least 14 weeks prior to screening and plan to continue taking the drug at the same dose and schedule during the trial

8. Adequate renal and hepatic function as defined:
Glomerular filtration rate ≥45 mL/min/1.73 m2 calculated by CKD-EPI
Alanine aminotransferase (ALT)≤3×ULN
Direct (conjugated) bilirubin ≤2×ULN

9. Patient must meet the following laboratory values at the screening visit:
Absolute Neutrophil Count ≥1.0×109/L
Hemoglobin >4.0 g/dL
Platelets ≥75×109/L

### Key Exclusion Criteria

Following are the key exclusion criteria:

1. Patients who have penile prosthetic implants or shunts or any other surgical procedure on the penis

2. Patients who have taken drugs/medications that may induce priapism (as per Appendix 5) over the 14 weeks pre-screening period

3. Patients who have received leuprolide acetate (Lupron) within 3 months before pre-screening.

4. Patients who had an erection lasting more than 12 hours over the 14 week pre-screening period

5. Patients who had an erection lasting more than 12 hours during the 12 weeks of the screening period

6. History of severe hypersensitivity reaction to other monoclonal antibodies, which in the opinion of the investigator may pose an increased risk of serious infusion reaction

7. Contraindication or hypersensitivity to any drug or metabolites from similar class as study drug or to any excipients of the study drug formulation

8. Use of therapeutic anticoagulants (prophylactic doses permitted) or antiplatelet therapy (other than aspirin or NSAIDs) within the 10 days prior to Week 1 Day 1 dosing. Note: prophylactic anticoagulant dose is permitted, as per local guidelines

9. Received a monoclonal antibody or immunomodulatory agent within 1 year of screening, or has documented immunogenicity to a prior biologic

10. Patient with any severe reaction to crizanlizumab, or discontinued crizanlizumab due to an AE

11. Patients should not be receiving another investigational trial within 30 days (or 5 half-lives of that agent, whichever is greater) prior to Screening or plans to participate in another investigational drug trial

12. Resting QTcF≥470 msec at pretreatment (baseline) or inability to determine QTc

13. Cardiac or cardiac repolarization abnormality, including any of the following:
History of myocardial infarction, angina pectoris, coronary artery bypass graft, or uncontrolled congestive heart failure within 6 months prior to starting study treatment
Clinically significant cardiac arrhythmias (e.g. ventricular tachycardia), complete left bundle branch block, high-grade AV block (e.g. bifascicular block, Mobitz type II and third degree AV block)

14. Current drug or alcohol abuse:
has a positive qualitative urine drug test at screening for cocaine, phencyclidine, or amphetamines
consumes >12 (for males) standard alcoholic beverages per week

15. Any documented history of a clinical stroke or intracranial hemorrhage, or an uninvestigated neurologic finding within the past 12 months before screening. Silent infarct only present on imaging is allowed

16. Clinically significant bleeding disorder

17. Planning to undergo a major surgical procedure during the duration of the study

18. Patient with active HIV infection (detectable viral load)

19. Patients with active Hepatitis B infection (HBsAg positive)

Note: Patients with antecedent but no active Hepatitis B (i.e. anti-HBc positive, HBsAg and HBV-DNA negative) are eligible

20. Patients with positive test for hepatitis C ribonucleic acid (HCV RNA)

Note: Patients in whom HCV infection resolved spontaneously (positive HCV antibodies without detectable HCV-RNA) or those that achieved a sustained virological response

### Study Treatment

Crizanlizumab (SEG101) at 5.0 mg/kg

On infusion day, the pharmacist or designated personnel prepares individual doses of crizanlizumab for subjects on a milligram per kilogram basis in a 100 mL infusion bag of a sterile 0.9% sodium chloride solution (0.9% Sodium Chloride Injection, USP) in accordance with the Pharmacy Manual. Study drug is administered over 30 minutes by IV infusion.

If a subject experiences a IRRs, he may receive pre-medication on subsequent dosing days as per institutional standard of care, at the discretion of the treating physician.

If a subject experiences a Grade 3 or 4 IRRs, the subject discontinues

### Concomitant Treatment

In general, the use of any concomitant medication/therapies deemed necessary for the care of the subject is permitted, except prohibited treatments. Hydroxy urea/Hydroxycarbamide or L-glutamine treatment is permitted if the subject has been prescribed any of these continuously over at least 14 weeks prior to the screening. The dosing should not be altered or terminated except for safety reasons. Erythropoietin-stimulating agents are also permitted to manage chronic symptomatic anemia with the same requirement for 6 months prior therapy. Aspirin, nonsteroidal anti-inflammatory drug (NSAIDs) and prophylactic doses (as per local guidelines) of anticoagulants are permitted, while other anti-platelets agents or anticoagulants at doses targeting therapeutic levels are prohibited. All approved forms of analgesia for pain are permitted per standard of care. Other approved medications for supportive care (antiemetics, anxiolytics, hypnotics, antihistamines) are permitted, including marinol (dronabinol).

Concomitant prophylactic treatment for SCD-related priapism is permitted provided the subject has been prescribed the same medication consistently for at least 14 weeks prior to screening as per inclusion criterion 7. Dosing should not be altered or terminated other than for safety during the duration of the study. In subjects not on prophylactic medications, treatment should not be initiated during the study. If a physician deems it necessary to terminate or alter treatment during the study, the monitor must be notified immediately to determine whether the subject may continue the study.

Although transfusion of cellular blood products is permitted, it is unclear how such transfusions would impact the PK/PD of crizanlizumab, so investigators are encouraged to obtain PK and PD samples before and after each transfusion session when possible. It should also be considered that the administration of products containing immunoglobulins (plasma, IVIG, anti-globulins) may also impact the efficacy of crizanlizumab, and optional PK and PD testing may also be performed prior to and following administration of such therapies. Although Endari™, the FDA-approved version of L-glutamine, is permitted, other over-the counter forms of L-glutamine are discouraged, as are other natural and herbal remedies (e.g. EvenFlo and/or products containing dang gui, Ligustrum root, ginseng root, white peony, corydalis, salvia, copodonosis, poria, jujube, Angelica sinensis, lovage) due the unproven efficacy and variable quality and composition of these products. Vitamin and mineral supplements (e.g. fish oil, folic acid, L-arginine, L-citrulline, magnesium, riboflavin, vitamin C, vitamin D, vitamin E, and zinc) are also permitted, though caution is advised when taking amounts exceeding 100% of the recommended daily allowance.

The use of other investigational drugs is prohibited during the study. In addition, the administration of monoclonal antibodies other than the investigational treatment is prohibited, due to the theoretical potential for cross-reactivity and/or overlapping toxicities with other monoclonal antibodies. If investigational agents or other monoclonal antibodies have been used in the past, they must have been discontinued at least 30 days or 5 half-lives of that agent, (whichever is greater) prior to the screening visit.

If a subject does not tolerate the protocol-specified dosing schedule, dose interruptions are either recommended or mandated in order to allow subjects to continue the study treatment until the next dose scheduled. Dose reductions are not allowed. If a subject experiences drug-induced toxicities, the subject should be closely monitored and a decision to continue or discontinue the subject from the study should be done at the next dose scheduled.

### Efficacy Assessment

Following are the key primary and secondary planned efficacy assessments:
Percent reduction in priapic events by 26 weeks of treatment
The rate of priapic events by 26 and 52 weeks of treatment
Percent reduction in acute priapic events by 26 and 52 weeks of treatment
The rate of complicated/uncomplicated

Priapism is defined as an unwanted or painful penile erection lasting at least 60 minutes. The end of the priapic event is the duration when the unwanted erection has resolved. This event is self-reported via an electronic reporting system, and this data should be collected throughout the study period. Primary efficacy endpoint is assessed by evaluating the percent reduction in priapic events by 26 weeks.

Acute priapic events are defined as an unwanted, painful erection that lasts more than 4 hours and need a visit to emergency room. Management of ischemic priapism require an aggressive and stepwise procedures to achieve prompt resolution. Aspiration/irrigation, in combination with intracavernous injections of α-agonist is usually the first-line therapy. Penile blood aspiration involves using a transglanular intracorporal angiocatheter insertion or a proximal penile shaft needle access. The percent reduction in acute priapic events from baseline by 26 and 52 weeks of treatment is assessed.

### Key Safety Assessments

Following are the key safety assessment for this study:
Monitoring of AEs/SAEs
Vital signs, Physical assessments
Hematology, chemistry, coagulation, urinalysis, Hepatitis markers
Presence of HIV Antibody (at screening only)
Cardiac assessments: ECGs and echocardiogram

### Other Assessments

Lower extremities examination for leg ulcer(s)
Symptoms and quality-of-life measurement by using the patient reported outcomes tools ASCQ-Me and PIP
Healthcare resource utilization (HRU)
Optional ultrasonography assessment of penile fibrosis

### Data Analysis

The primary endpoint of the study is the percent reduction from baseline inpriapic events by 26 weeks (i.e. up to pre-infusion Week 27, Day 1). Efficacy endpoints are analyzed using all FAS (Full Analysis Set) subjects who have completed treatment. It has been hypothesized that crizanlizumab treatment reduces the priapic events by at least 25% in SCD subjects with priapism.

Percent reduction from baseline in priapic events are tested using a nonparametric test (i.e. Wilcoxon's Sign Rank test) along with Hodges-Lehmann estimate of median percent reduction Number of priapic events and percent reduction from baseline by Week 26 are summarized descriptively.

Demonstration of significant percent reduction from baseline in priapic events is evaluated using the following hypothesis:

H0: p<0.25, where p is the percent reduction in priapic events by 26 weeks.

H1: p≥0.25, where p is the percent reduction in priapic events by 26 weeks.

If the above null hypothesis is rejected, significant reduction in priapic events is demonstrated statistically. Percent reduction from baseline in priapic events is tested using a nonparametric test (i.e. Wilcoxon's Sign Rank test). Hodges-Lehmann estimate of median percent reduction in priapic

events is also reported, along with corresponding 95% confidence intervals. The significance of the efficacy endpoints is assessed at α=0.05 level.

The primary analysis is performed on all FAS patients who have completed 26 weeks on treatment.

Number of priapic events is summarized at baseline and by Week 26, and percent reduction from baseline by Week 26 is summarized by mean, standard deviation, median, minimum and maximum. In addition, subgroup analysis of the primary endpoint is also performed based on the number of priapic events categories (i.e. 7-13, 14-21, ≥22) at baseline. The priapic events categories may be re-grouped to ensure that there is adequate number of subjects in each category for analysis. Additional analyses of the primary endpoint is also performed for the subgroups of subjects with stuttering and acute priapism. The FAS consists of all subjects to whom the study treatment has been assigned and who received at least 1 dose of a study treatment and have at least 1 post-baseline assessment.

As part of the supportive analysis, the primary analysis is repeated on all subjects in FAS. In addition the annualized rate of priapic events are summarized for all subjects in FAS that have completed treatment. Three additional supportive analyses are also performed, the total number of priapic episodes in the screening period (12 weeks baseline) is compared to the total number of priapic episodes occurring in the first 12 weeks on treatment (0-12 weeks) using a nonparametric test (i.e. Wilcoxon's Sign Rank test). A similar analysis is performed to compare to the total number of priapic episodes occurring in the last 12 weeks on treatment (15-26 weeks). Finally an analysis using a mixed effects regression model is performed using median event counts within prespecified time windows.

Similar analyses on percent reduction in acute priapic events for subjects is conducted, as was completed for primary end-point. Descriptive summary statistics are presented for the rate of events and number of priapic events at Baseline, by Week 26 and by Week 52. In addition the rate of uncomplicated VOC events and VOC events is summarized. Safety analyses is based on the safety analysis set. Adverse events are summarized by system organ class and or preferred term, seriousness, CTCAE (Common Terminology Criteria for Adverse events) grade based severity, type of adverse event, relation to study treatment. Other safety assessments (e.g. ECG, vital signs) are summarized by treatment group.

As of March 2020, four patients in the SPARTAN trial had undergone 10 weeks of treatment with crizanlizumab. Our preliminary findings are the following:
Patient 1 baseline was 84 priapic episodes in 26 weeks (ca 3.2 episodes per week). At week 10 of treatment, the number of priapic episodes recorded for Patient 1 was 30 (ca 3 episodes per week), corresponding to a reduction of 7% in the frequency of priapic episodes.
Patient 2 baseline was 173 priapic episodes in 26 weeks (ca 6.7 episodes per week). At week 10 of treatment, the number of priapic episodes recorded for Patient 2 was 50 (ca 5 episodes per week), corresponding to a reduction of 25% in the frequency of priapic episodes.
Patient 3 baseline was 56 priapic episodes in 26 weeks (ca 2.2 episodes per week). At week 10 of treatment, the number of priapic episodes recorded for Patient 3 was 11 (ca 1.1 episodes per week), corresponding to a reduction of 49% in the frequency of priapic episodes.
Patient 4 baseline was 10 priapic episodes in 26 weeks (ca 0.4 episodes per week). At week 10 of treatment, the number of priapic episodes recorded for Patient 4 was 2 (ca 0.2 episodes per week), corresponding to a reduction of 48% in the frequency of priapic episodes.

Based on these 4 patients, over the 10 weeks of treatment, the number of priapic episodes per patient per week went from 3.1 to 2.3 corresponding to a 25% reduction in the frequency of priapic episodes.

The results indicate efficiency of the treatment with crizanlizumab in the treatment of priapism in SCD patients.

Sequences

SEQ ID NO: 1 P-selectin amino acid sequence
WTYHYSTKAYSWNISRKYCQNRYTDLVAIQNKNEIDYLNKVLPYYSSYYWIGIRKNNKTWTWVGTKKALT
NEAENWADNEPNNKRNNEDCVEIYIKSPSAPGKWNDEHCLKKKHALCYTASCQDMSCSKQGECLETIGNY
TCSCYPGFYGPECEYVRECGELELPQHVLMNCSHPLGNFSFNSQCSFHCTDGYQVNGPSKLECLASGIWT
NKPPQCLAAQCPPLKIPERGNMTCLHSAKAFQHQSSCSFSCEEGFALVGPEVVQCTASGVWTAPAPVCK
SEQ ID NO: 2 CDR light chain amino acid sequence
KASQSVDYDGHSYMN
SEQ ID NO: 3 CDR light chain amino acid sequence
AASNLES
SEQ ID NO: 4 CDR light chain amino acid sequence
QQSDENPLT
SEQ ID NO: 5 Mature light chain variable region amino acid sequence
DIQMTQSPSSLSASVGDRVTITCKASQSVDYDGHSYMNWYQQKPGKAPKLLIYAASNLESGVPSRFSGSG
SGTDFTLTISSLQPEDFATYYCQQSDENPLTFGGGTKVEIKR
SEQ ID NO: 6 CDR heavy chain amino acid sequence
SYDIN
SEQ ID NO: 7 CDR heavy chain amino acid sequence
WIYPGDGSIKYNEKFKG
SEQ ID NO: 8 CDR heavy chain amino acid sequence
RGEYGNYEGAMDY
SEQ ID NO: 9 Mature heavy chain variable region amino acid sequence
QVQLVQSGAEVKKPGASVKVSCKVSGYTFTSYDINWVRQAPGKGLEWMGWIYPGDGSIKYNEKFKGRVTM
TVDKSTDTAYMELSSLRSEDTAVYYCARRGEYGNYEGAMDYWGQGTLVTVSS
SEQ ID NO: 10 Human Kappa constant region amino acid sequence
TVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO: 11 IgG₂ constant region amino acid sequence
ASTKGPSVFPLAPCSRSTSESTAALGCLVKDYFPEPVTVSWNSGALTSGVHTFPAVLQSSGLYSLSSVVT
VTSSNFGTQTYTCNVDHKPSNTKVDKTVERKCCVECPPCPAPPVAGPSVFLFPPKPKDTLMISRTPEVIC
VVVDVSHEDPEVQFNWYVDGMEVHNAKTKPREEQFNSTFRVVSVLTVVHQDWLNGKEYKCKVSNKGLPAP
IEKTISKTKGQPREPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
SEQ ID NO: 12 Light chain mature amino acid sequence
DIQMTQSPSSLSASVGDRVTITCKASQSVDYDGHSYMNWYQQKPGKAPKLLIYAASNLESGVPSRFSGSG
SGTDFTLTISSLQPEDFATYYCQQSDENPLTFGGGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSPVT
KSFNRGEC
SEQ ID NO: 13 Heavy chain mature amino acid sequence
QVQLVQSGAEVKKPGASVKVSCKVSGYTFTSYDINWVRQAPGKGLEWMGWIYPGDGSIKYNEKFKGRVTM
TVDKSTDTAYMELSSLRSEDTAVYYCARRGEYGNYEGAMDYWGQGTLVTVSSASTKGPSVFPLAPCSRST
SESTAALGCLVKDYFPEPVTVSWNSGALTSGVHTFPAVLQSSGLYSLSSVVTVTSSNFGTQTYTCNVDHK
PSNTKVDKTVERKCCVECPPCPAPPVAGPSVFLFPPKPKDTLMISRIPEVTCVVVDVSHEDPEVQFNWYV
DGMEVHNAKTKPREEQFNSTFRVVSVLTVVHQDWLNGKEYKCAVSNKGLPAPIEKTISKTKGQPREPQVY
TLPPSREEMTKNQVSLTCLVKGFYPSDIAVEWESNGQPENNYKTTPPMLDSDGSFFLYSKLTVDKSRWQQ
GNVFSCSVMHEALHNHYTQKSLSLSPGK
SEQ ID NO: 14 Light chain complete amino acid sequence
MESQTQVFVYMLLWLSGVDGDIQMTQSPSSLSASVGDRVTITCKASQSVDYDGHSYMNWYQQKPGKAPKL
LIYAASNLESGVPSRFSGSGSGTDFTLTISSLQPEDFATYYCQQSDENPLTFGGGTKVEIKRTVAAPSVF
IFPPSDEQLKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID NO: 15 Heavy chain complete amino acid sequence
MKCSWVIFFLMAVVTGVNSQVQLVQSGAEVKKPGASVKVSCKVSGYTFTSYDINWVRQAPGKGLEWMGWI
YPGDGSIKYNEKFKGRVTMTVDKSTDTAYMELSSLRSEDTAVYYCARRGEYGNYEGAMDYWGQGTLVTVS
SASTKGPSVFPLAPCSRSTSESTAALGCLVKDYFPEPVTVSWNSGALTSGVHTFPAVLQSSGLYSLSSVV
TVTSSNFGTQTYTCNVDHKPSNTKVDKTVERKCCVECPPCPAPPVAGPSVFLFPPKPKDTLMISRTPEVT
CVVVDVSHEDPEVQFNWYVDGMEVHNAKTKPREEQFNSTFRVVSVLTVVHQDWLNGKEYKCAVSNKGLPA
PIEKTISKTKGQPREPQVYTLPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGK
SEQ ID NO: 18 Complete light variable region amino acid sequence
MESQTQVFVYMLLWLSGVDGDIQMTQSPSSLSASVGDRVTITCKASQSVDYDGHSYMNWYQQKPGKAPKL
LIYAASNLESGVPSRFSGSGSGTDFTLTISSLQPEDFATYYCQQSDENPLTFGGGTKVEIKR
SEQ ID NO: 19 Complete heavy variable region amino acid sequence
MKCSWVIFFLMAVVTGVNSQVQLVQSGAEVKKPGASVKVSCKVSGYTFTSYDINWVRQAPGKGLEWMGWI
YPGDGSIKYNEKFKGRVTMTVDKSTDTAYMELSSLRSEDTAVYYCARRGEYGNYEGAMDYWGQGTLVTVS
S
SEQ ID NO: 23 IgG₂ constant region amino acid sequence with a one amino
acid residue mutation to reduce complement activation
ASTKGPSVFPLAPCSRSTSESTAALGCLVKDYFPEPVTVSWNSGALTSGVHTFPAVLQSSGLYSLSSVVT
VTSSNFGTQTYTCNVDHKPSNTKVDKTVERKCCVECPPCPAPPVAGPSVFLFPPKPKDTLMISRTPEVIC
VVVDVSHEDPEVQFNWYVDGMEVHNAKTKPREEQFNSTFRVVSVLTVVHQDWLNGKEYKCAVSNKGLPAP
IEKTISKTKGQPREPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK

1. A method of treating priapism in a patient in need of such treatment, comprising administering a pharmaceutically effective amount of an anti-P-selectin antibody or a binding fragment thereof to said patient.

2. The method according to claim 1, wherein the patient is suffering from Sickle Cell Disease (SCD).

3. The method according to claim 1, wherein the anti-P-selectin antibody is crizanlizumab or a binding fragment thereof.

4. The method according to claim 1, wherein the patient receives co-treatment with hydroxyurea, hydroxycarbamide, L-glutamine, Erythropoietin-stimulating agents, acetylsalicylic acid, nonsteroidal anti-inflammatory drug, leuprolide, or anticoagulants at prophylactic doses, or any combination of two or more thereof.

5. The method according to claim 3, wherein the patient receives co-treatment with hydroxyurea, hydroxycarbamide, L-glutamine, Erythropoietin-stimulating agents, acetylsalicylic acid, nonsteroidal anti-inflammatory drug, leuprolide, or anticoagulants at prophylactic doses, or any combination of two or more thereof.

6. The method of claim 1 wherein the treatment is administered as monotherapy.

7. The method according to claim 3 wherein the treatment is administered as monotherapy with crizanlizumab.

8. The method according to claim 3, wherein crizanlizumab or a binding fragment thereof, is administered 5 mg/kg or 7.5 mg/kg per treatment.

9. The method of claim 3, wherein crizanlizumab or a binding fragment thereof, is administered 5 mg/kg or 7.5 mg/kg monthly.

10. The method of claim 3, wherein crizanlizumab or a binding fragment thereof, is administered in a loading dose followed by a maintenance dose.

11. The method according to claim 10, wherein the loading dose is 5 mg/kg or 7.5 mg/kg and is administered twice in a time interval of 2 weeks+/−3 days, and the maintenance dosage is then administered 4 weeks+/−3 days after the second loading dose administration and then at regular intervals of 4 weeks+/−3 days.

12. The method according to claim 1, where the priapism treated is defined as an unwanted or painful penile erection lasting at least 60 minutes.

13. The method according to claim 3, where the priapism treated is defined as an unwanted penile erection lasting at least 60 minutes.

14. The method according to claim 8, where the priapism treated is defined as an unwanted penile erection lasting at least 60 minutes.

15. The method according to claim 1, wherein the treatment is a monotherapy with crizanlizumab.

16. The method according to claim 1, where the anti-P-Selectin antibody or binding fragment thereof comprises a heavy chain variable region comprising three CDRs comprising, consisting essentially of or consisting of SEQ ID NO: 6, SEQ ID NO: 7, and SEQ ID NO: 8, respectively and a light chain variable region comprising three CDRs comprising, consisting essentially of or consisting of SEQ ID NO: 2, SEQ ID NO: 3, and SEQ ID NO: 4, respectively.


SEQUENCE LISTING (23 sequences)

SEQ ID NO: 1 - protein, 279 aa, Homo sapiens
WTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCK

SEQ ID NO: 2 - protein, 15 aa, Artificial Sequence
  CDR light chain amino acid sequence
KASQSVDYDGHSYMN

SEQ ID NO: 3 - protein, 7 aa, Artificial Sequence
  CDR light chain amino acid sequence
AASNLES

SEQ ID NO: 4 - protein, 9 aa, Artificial Sequence
  CDR light chain amino acid sequence
QQSDENPLT

SEQ ID NO: 5 - protein, 112 aa, Artificial Sequence
  Mature light chain variable region amino acid sequence
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 6 - protein, 5 aa, Artificial Sequence
  CDR heavy chain amino acid sequence
SYDIN

SEQ ID NO: 7 - protein, 17 aa, Artificial Sequence
  CDR heavy chain amino acid sequence
WIYPGDGSIKYNEKFKG

SEQ ID NO: 8 - protein, 13 aa, Artificial Sequence
  CDR heavy chain amino acid sequence
RGEYGNYEGAMDY

SEQ ID NO: 9 - protein, 122 aa, Artificial Sequence
  Mature heavy chain variable region amino acid sequence
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSS

SEQ ID NO: 10 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 218 aa, Artificial Sequence
  Light chain mature amino acid sequence
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 448 aa, Artificial Sequence
  Heavy chain mature amino acid sequence
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 238 aa, Artificial Sequence
  Light chain complete amino acid sequence
MESQTQVFVYMLLWLSGVDGDIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 467 aa, Artificial Sequence
  Heavy chain complete amino acid sequence
MKCSWVIFFLMAVVTGVNSQVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16-17 - skipped (2 entries)

SEQ ID NO: 18 - protein, 132 aa, Artificial Sequence
  Complete light variable region amino acid sequence
MESQTQVFVYMLLWLSGVDGDIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 19 - protein, 141 aa, Artificial Sequence
  Complete heavy variable region amino acid sequence
MKCSWVIFFLMAVVTGVNSQVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSS

SEQ ID NO: 20-22 - skipped (3 entries)

SEQ ID NO: 23 - protein, 326 aa, Artificial Sequence
  IgG2 constant region amino acid sequence with a one amino acid residue mutation to reduce complement activation
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK